(19) United States
(12) Patent Application Publication
(10) Pub. No.: US 2024/0123026 A1
(43) Pub. Date: Apr. 18, 2024

(54) Actinohivin Variant Polypeptides and Related Methods

(71) Applicant: University of Louisville Research Foundation, Inc., Louisville, KY (US)

(72) Inventors: Nobuyuki Matoba, Owensboro, KY (US); Matthew Dent, Louisville, KY (US)

(21) Appl. No.: 18/277,684

(22) PCT Filed: Mar. 4, 2022
(86) PCT No.: PCT/US2022/070961

§ 371 Date: Aug. 17, 2023

Related U.S. Application Data
(60) Provisional application No. 63/156,715, filed on Mar. 4, 2021.

(51) Int. Cl. A61K 38/16; A61P 35/00

(52) U.S. Cl. CPC A61K 38/168; A61P 35/00

(57) ABSTRACT

The treatment of ovarian cancers (e.g., epithelial ovarian cancers), which are sensitive to polypeptides comprising an actinohivin or a variant thereof (e.g., AvFc), is described. Methods for killing ovarian cancer cells (e.g, epithelial ovarian cancer cells) are also provided.

### RELATED APPLICATION(S)

This application claims the benefit of U.S. Provisional Application No. 63/156,715, filed on Mar. 4, 2021. The entire teachings of the above application(s) are incorporated herein by reference.

### GOVERNMENT SUPPORT

This invention was made with government support under 1R21CA216447-01 from NIH/NCI and R21/R33 AI088585 from NIH/NIAID. The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:
a) File name: 56001010001PCT_Sequence_Listing_ST25.txt; created Feb. 23, 2022, 19,000 Bytes in size.

### BACKGROUND

Ovarian cancer (OVCA), in particular, epithelial ovarian cancer (EOC), is one of the deadliest gynecological cancers, ranking fifth in cancer death among women. EOC typically begins as small, borderline epithelial tumors on either the surface of the ovary, the fallopian tubes, or the mesothelium lining of the peritoneal cavity. These tumors grow and become well differentiated before metastasizing, primarily to the abdominal cavity but rarely to the lungs, liver, and brain. According to the National Cancer Institute (NCI) Surveillance, Epidemiology, and End Results (SEER) Program, the overall 5-year-survival rate in the United States is 48.3% as of 2016, largely driven by the dismal survival rate (30.5%) of late-stage disease. The age-adjusted mortality rate and rate of new cases of OVCA is 6.8 per 100,000 per year and 10.5 per 100,000 per year, respectively, slightly above that of the next deadliest gynecological cancer, uterine cancer, with a mortality rate of 5.0 per 100,000 per year. This translates to an increase in 2016 of 22,530 new patients and 13,980 deaths. While the numbers of new cases and deaths are trending downwards slowly over the past 20 years, the prognosis of patients, especially those with late-stage disease, remains poor. This is largely due to ineffective population-based screening, innocuous presentation, and the lack of effective second line therapies for chemo-resistant disease. Although patients generally respond very well to the primary treatment, the vast majority of women (75%) will experience disease recurrence that is incurable due to chemo-resistance.

Ovarian cancer standards of care and their shortcomings. Primary debulking surgery followed by chemotherapy has been the first-line standard of care for EOC for decades. Surgery for advanced disease consists of total abdominal hysterectomy, bilateral salpingo-oophorectomy, and omentectomy, though patients with low grade disease can opt for a fertility conservation strategy. The vast majority of patients will also receive chemotherapy consisting of a platinum-based drug, most often carboplatin, and a taxane, such as paclitaxel. No residual disease following primary therapy is the most important prognostic indicator. While this is achievable for most patients regardless of disease stage, nearly all will inevitably experience fatal chemo-resistant disease. Treatment options at this stage are limited based on the platinum-free interval (the length of time between platinum drug treatments) of the patient and the amenability of the subsequent disease to secondary debulking surgery, though the likelihood of survival is poor regardless. For patients who have gone through the first-line standard of care, a greater benefit has been demonstrated with the use of long-term maintenance therapy, which consists of chemotherapeutics or biologics given after no residual disease is achieved to prolong survival. FDA approval of bevacizumab and poly ADP ribose polymerase (PARP) inhibitors has expanded the availability of maintenance therapy and improved progression-free survival; however, current clinical data have not demonstrated significant increases in overall survival and these drugs are associated with significant adverse events. Furthermore, there is no FDA-approved targeted immunotherapy for EOC, and trials with checkpoint inhibitors have not been conclusive.

### SUMMARY

There is a critical need for novel anti-ovarian cancer drugs, including complementary therapies for curing the disease, prolonging progression, and improving survival in patients.

The subject matter disclosed herein is based, in part, on the discovery that a fusion protein consisting of Avaren lectin and human IgG1 Fc (AvFc) targets a unique biomarker of epithelial ovarian cancer (EOC). AvFc is selective for oligomannose glycans overrepresented on the surface of cancer cells. AvFc mediates anti-cancer activities including antibody-dependent cell-mediated cytotoxicity (ADCC).

In one aspect, the disclosure provides a method of killing an ovarian cancer (OVCA) cell, the method comprises contacting the OVCA cell with a polypeptide comprising an actinohivin or a variant thereof.

In another aspect, the disclosure provides a method of inducing antibody-dependent cell-mediated cytotoxicity (ADCC) in an OVCA cell, the method comprises contacting the OVCA cell with a polypeptide comprising an actinohivin or a variant thereof.

In another aspect, the disclosure provides a method of treating OVCA in a subject in need thereof, the method comprises administering to the subject a therapeutically effective amount of a polypeptide comprising an actinohivin or a variant thereof.

In another aspect, the disclosure provides use of a polypeptide in manufacture of a medicament for treating OVCA in a subject in need thereof, wherein the polypeptide comprises an actinohivin or a variant thereof, and wherein the treatment comprises administering to the subject an effective amount of the polypeptide.

In another aspect, the disclosure provides a polypeptide for use in treating OVCA in a subject in need thereof, wherein the polypeptide comprises an actinohivin or a variant thereof, and wherein the treatment comprises administering to the subject an effective amount of the polypeptide.

In another aspect, the disclosure provides a method of reducing tumor size in a subject having OVCA, the method comprises administering to the subject an effective amount of a polypeptide comprising an actinohivin or a variant thereof, wherein the administering of the polypeptide reduces the tumor size.

In another aspect, the disclosure provides use of a polypeptide in manufacture of a medicament for reducing tumor size in a subject having OVCA, wherein the polypeptide comprises an actinohivin or a variant thereof, and wherein the treatment comprises administering to the subject an effective amount of the polypeptide.

In another aspect, the disclosure provides a polypeptide for use in reducing tumor size in a subject having OVCA, wherein the polypeptide comprises an actinohivin or a variant thereof, and wherein the treatment comprises administering to the subject an effective amount of the polypeptide.

In another aspect, the disclosure provides a method of treating EOC in a subject in need thereof, the method comprises administering to the subject an effective amount of a polypeptide comprising an amino acid sequence set forth in SEQ ID NO:16.

In another aspect, the disclosure provides use of a polypeptide in manufacture of a medicament for treating EOC in a subject in need thereof, wherein the polypeptide comprises an amino acid sequence set forth in SEQ ID NO:16, and wherein the method comprises administering to the subject an effective amount of the polypeptide.

In another aspect, the disclosure provides a polypeptide for use in treating EOC in a subject in need thereof, wherein the polypeptide comprises an amino acid sequence set forth in SEQ ID NO:16, and wherein the method comprises administering to the subject an effective amount of the polypeptide.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D show activity of Avaren-Fc (AvFc) against ovarian cancer (OVCA). FIGS. 1A-1B are immunohistochemistry of human epithelial ovarian cancer (EOC) tissue sections demonstrating oligomannose-dependent binding of AvFc to human OVCA tissue. FIG. 1A shows malignant and adjacent tissue stained with AvFc. Little to no staining is seen in the adjacent tissue while the malignant tissue is highly bound by AvFc. AvFc clearly delineates malignant from normal adjacent tissue as seen by the level of DAB staining. FIG. 1B shows malignant and adjacent tissue stained with a non-sugar-binding mutant, AvFc^{lec-}. No binding is seen in either case, indicating that AvFc's binding to human OVCA tissue is oligomannose-dependent. FIG. 1C shows binding of AvFc to OVCA cell lines A2780, SKOV3, ID8, and ID8-VEGF-DEFB29 as determined by single color flow cytometry. Y-axis shows percentage of FITC⁺ as determined by gating against background fluorescence. FIG. 1D shows antibody-dependent cell-mediated cytotoxicity (ADCC) activity of AvFc against OVCA cell lines with a luciferase-based reporter cell line assay. AvFc potently induces ADCC against all 4 cancer cell lines with the highest degrees of activity seen against the ID8 and ID8-VEGF-DEFB29 cell lines.

FIG. 2 shows binding of AvFc to murine and human OVCA cell lines determined by flow cytometry. Briefly, AvFc was incubated with each cell line at concentrations ranging from 1.3 to 130 nM, followed by detection of AvFc with a goat antihuman Fc-FITC conjugate. A non-sugar-binding mutant, AvFc^{lec-}, was used as a negative control.

FIGS. 3A-3B are representative fluorescent micrographs of AvFc binding to the murine OVCA cell line ID8 (FIG. 3A) and the human cell line A2780 (FIG. 3B).

FIG. 4 shows mouse ID8 OVCA challenge model. Female C57bl/6 mice were intraperitoneally (i.p.) challenged with two million ID8 cells per animal. Starting from 7 days post tumor challenge, mice were treated i.p. every 2 days with either 25 mg/kg AvFc or vehicle control for 28 days (Days 7-35; 15 doses total), and survival was monitored after treatment cessation until all animals died or reached a euthanasia point (>35 g body weight). Survival curves were determined to be significantly different by the long-rank test (P=0.0048; GraphPad Prism 8 software).

### DETAILED DESCRIPTION

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The disclosure is not limited by the illustrated ordering of acts or events, as some acts may occur in different orders and/or concurrently with other acts or events. Furthermore, not all illustrated acts, steps or events are required to implement a methodology in accordance with the disclosure. Many of the techniques and procedures described, or referenced herein, are well understood and commonly employed using conventional methodology by those skilled in the art.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

The term “polypeptide” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L-and/or D-amino acid, for example, common a-amino acids (e.g., alanine, glycine, valine), non-a-amino acids (e.g., b-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-a-amino group substitution), if desired. In addition, a protein, peptide or polypeptide can be an analog of a known and/or naturally-occurring peptide, for example, a peptide analog having conservative amino acid residue substitution(s).

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Carrier” refers to a diluent, adjuvant, excipient, or vehicle with which the antibody of the disclosure is administered. Such vehicles may be liquids, such as water and oils, including those of petroleum, animal, vegetable or synthetic origin, such as peanut oil, soybean oil, mineral oil, sesame oil and the like. For example, histidine, sodium chloride, and sucrose may be used to formulate a polypeptide of the disclosure. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, See especially pp. 958-989.

A description of example embodiments follows.

### Methods of Killing Cancer Cells

In one aspect, the disclosure provides a method of killing an ovarian cancer (OVCA) cell, the method comprises contacting the OVCA cell with a polypeptide comprising an actinohivin or a variant thereof.

In another aspect, the disclosure provides a method of inducing antibody-dependent cell-mediated cytotoxicity (ADCC) in an OVCA cell, the method comprises contacting the OVCA cell with a polypeptide comprising an actinohivin or a variant thereof.

In some embodiments, contacting the OVCA cell with the polypeptide results in at least about 50% induction of ADCC compared to the baseline level (e.g., pretreatment), for example, resulting in at least about: 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, 15-fold, 15.5-fold, 16-fold, 16.5-fold, 17-fold, 17.5-fold, 18-fold, 18.5-fold, 19-fold, 19.5-fold, or 20-fold induction of ADCC. In some embodiments, contacting the OVCA cell with the polypeptide results in about 1.5-20 fold induction of ADCC compared to the baseline level, for example, about: 1.5-19.5 fold, 2-19.5 fold, 2-19 fold, 2.5-19 fold, 2.5-18.5 fold, 3-18.5 fold, 3-18 fold, 3.5-18 fold, 3.5-17.5 fold, 4-17.5 fold, 4-17 fold, 4.5-16.5 fold, 5-16.5 fold, 5-16 fold, 5.5-16 fold, 5.5-15.5 fold, 6-15.5 fold, 6-15 fold, 6.5-15 fold, 6.5-14.5 fold, 7-14.5 fold, 7-14 fold, 7.5-14 fold, 7.5-13.5 fold, 8-13.5 fold, 8-13 fold, 8.5-13 fold, 8.5-12.5 fold, 9-12.5 fold, 9-12 fold, 9.5-12 fold, 9.5-11.5 fold, 10-11.5 fold or 10-11 fold induction of ADCC.

### Polypeptides

In some embodiments, the polypeptide comprises a wildtype actinohivin amino acid sequence (e.g., SEQ ID NO:1) or a variant thereof. Actinohivin is a sugar-binding protein exhibiting an anti-human immunodeficiency virus activity, which was originally identified and isolated from the actinomycete K97-0003 strain (Chiba et al., Biochem Biophys Res Commun. 282:595-601 (2001)).

In particular embodiments, the polypeptide comprises a wildtype actinohivin amino acid sequence (e.g., SEQ ID NO:1).

In certain embodiments, the polypeptide comprises a variant of a wildtype actinohivin amino acid sequence (e.g., SEQ ID NO:1). As used herein, the term “variant” refers to a polypeptide comprising an amino acid sequence that has at least about 70% sequence identity to a reference sequence, i.e., a wild type actinohivin.

In certain embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:1-15. In particular embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:1-13.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:1. In particular embodiments, the polypeptide comprises an amino acid sequence set forth in SEQ ID NO:1.

In certain embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:2-15. In particular embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:2-13.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:2. In particular embodiments, the polypeptide comprises an amino acid sequence set forth in SEQ ID NO:2.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:3. In particular embodiments, the polypeptide comprises an amino acid sequence set forth in SEQ ID NO:3.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:4. In particular embodiments, the polypeptide comprises an amino acid sequence set forth in SEQ ID NO:4.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:5. In particular embodiments, the polypeptide comprises an amino acid sequence set forth in SEQ ID NO:5.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:6. In particular embodiments, the polypeptide comprises an amino acid sequence set forth in SEQ ID NO:6.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:7. In particular embodiments, the polypeptide comprises an amino acid sequence set forth in SEQ ID NO:7.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:8. In particular embodiments, the polypeptide comprises an amino acid sequence set forth in SEQ ID NO:8.

In particular embodiments, the polypeptide comprises an amino acid sequence that is at least about 75% (e.g., at least about: 80, 85, 90, 91, 92, 93, 94, 95, 96, 97, 98 or 99%) identical to SEQ ID NO:9. In some embodiments, the polypeptide comprises an amino acid sequence set forth in SEQ ID NO:9.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:10. In particular embodiments, the polypeptide comprises an amino acid sequence set forth in SEQ ID NO:10.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:11. In particular embodiments, the polypeptide comprises an amino acid sequence set forth in SEQ ID NO:11.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:12. In particular embodiments, the polypeptide comprises an amino acid sequence set forth in SEQ ID NO:12.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:13. In particular embodiments, the polypeptide comprises an amino acid sequence set forth in SEQ ID NO:13.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:14. In particular embodiments, the polypeptide comprises an amino acid sequence set forth in SEQ ID NO:14.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:15. In particular embodiments, the polypeptide comprises an amino acid sequence set forth in SEQ ID NO:15.

In some embodiments, the polypeptide is modified, e.g., with “GASDALIE” and/or “GnGn” modifications. “GASDALIE” modification refers to G236A/S239D/A330L/I332E mutations in the IgG1 Fc domain (see, e.g., Ahmed, A. A., et al., Journal of structural biology 194(1):78-89 (2016)). “GnGn” modification refers to Fc glycan modifications to contain primarily terminal GlcNAc residues and lack plant-specific glycans (Strasser, R., et al., Plant Biotechnology Journal 6(4):392-402 (2008)).

In some embodiments, the polypeptide further comprises a fragment crystallizable domain of an antibody (Fc), a fragment antigen-binding domain of an antibody (Fab) or a single chain variable fragment of an antibody (scFv). In certain embodiments, the polypeptide further comprises an Fab. In particular embodiments, the polypeptide further comprises a scFv. In some embodiments, the polypeptide further comprises an Fc. In certain embodiments, the polypeptide comprises the high-mannose glycan-binding (actinomycete-derived, oligomannose-binding) lectin Avaren and IgG1 Fc (fragment crystallizable region (Fc) of human immunoglobulin G1) (the “lectibody” Avaren-Fc (AvFc)).

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:16. In particular embodiments, the polypeptide comprises an amino acid sequence set forth in SEQ ID NO:16.

In certain embodiments, the Fc region of the polypeptide is modified to optimize Pharmacokinetics (PK) and/or Pharmacodynamics (PD) properties, such as M428L and N434S mutations for extended plasma half-life (M. R. Gaudinski, et al., PLoS Med. 15 (2018), e1002493).

In particular embodiments, the polypeptide is conjugated to a cytotoxic chemical, a DNA-damaging agent, a radioisotope or a combination thereof. In some embodiments, the polypeptide is conjugated to a cytotoxic chemical. In certain embodiments, the cytotoxic chemical comprises a tubulin inhibitor (e.g., a maytansinoid or an auristatin). In certain embodiments, the polypeptide is conjugated to a DNA-damaging agent. In some embodiments, the DNA-damaging agent comprises a calicheamicin. In particular embodiments, the polypeptide is conjugated to a radioisotope. In certain embodiments, the radioisotope is lutetium-177.

In some embodiments, the polypeptide is plant-produced. In other embodiments, the polypeptide is produced in mammalian cells (e.g., CHO cells).

In certain embodiments, the polypeptide is an isolated polypeptide. An “isolated” polypeptide is one that has been identified and separated and/or recovered from a component of its natural environment. Contaminant components of its natural environment are materials that would interfere with diagnostic and/or therapeutic uses for the polypeptide, for example, enzymes, hormones, and other proteinaceous or nonproteinaceous solutes. An “isolated” polypeptide encompasses polypeptides that are isolated to a higher purity, such as polypeptides that are 80%, 81%, 82%, 83%, 84%, 85%, 86%, 87%, 88%, 89%, 90%, 91%, 92%, 93%, 94%, 95%, 96%, 97%, 98%, 99% or 100% pure.

### High-Mannose-Type Glycan Epitopes

In certain embodiments, the polypeptide is highly selective to (or specifically binds) malignant cells over noncancerous or normal healthy cells. The term “specifically binding” or “specifically binds” refers to preferential interaction, i.e., significantly higher binding affinity, between the polypeptide and a malignant cell relative to a normal healthy cell.

In some embodiments, the binding affinity between the polypeptide and a malignant OVCA cell (e.g., an epithelial ovarian cancer (EOC) cell) relative to a normal healthy cell is at least about 2-fold higher, for example, at least about: 2.5, 3, 3.5, 4, 4.5, 5, 5.5, 6, 6.5, 7, 7.5, 8, 8.5, 9, 9.5, 10-fold higher. In certain embodiments, the binding affinity between the polypeptide and a malignant OVCA cell (e.g., an EOC cell) relative to a normal healthy cell is at least about 10-fold higher, for example, at least about: 15, 20, 25, 30, 35, 40, 45, 50, 55, 60, 65, 70, 75, 80, 85, 90, 95 or 100-fold higher. In particular embodiments, the binding affinity between the polypeptide and a malignant OVCA cell (e.g., an EOC cell) relative to a normal healthy cell is about 2-10 times higher, for example about: 2.5-10, 2.5-9.5, 2.5-9, 3-9, 3-8.5, 3.5-8.5, 3.5-8, 4-8, 4-7.5, 4.5-7, 4.5-6.5, 5-6.5, 5-6 or 5.5-6 times higher. In some embodiments, the binding affinity between the polypeptide and a malignant OVCA cell (e.g., an EOC cell) relative to a normal healthy cell is about 10-100 times higher, for example, about: 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90 or 90-100 times higher.

In particular embodiments, the EC₅₀ of a polypeptide of the disclosure to an OVCA cell (e.g., an EOC cell) is about 1-10 nM, for example, about: 1, 2, 3, 4, 5, 6, 7, 8, 9, 10, 1-9, 2-9, 2-8, 3-8, 3-7, 4-7, 4-6 or 5-6 nM.

In certain embodiments, the polypeptide specifically binds a high-mannose-type glycan epitope.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come into close proximity in a three-dimensional space through the folding of the protein molecule.

The term “high-mannose-type glycan” refers to asparagine-linked glycan (N-glycan) containing 5-9 terminal mannose residues attached to the chitobiose (GlcNAc₂) core. High-mannose glycans are formed and attached to newly synthesized nascent polypeptides containing asparagine-X-serine/threonine sequences, where X can be any amino acid except for proline, in the endoplasmic reticulum of eukaryotic cells. These glycans are then typically processed and matured into complex-type glycans containing fewer mannose residues as the nascent polypeptides undergo the secretory pathway through the Golgi apparatus. As a consequence, few high-mannose glycans remain attached to proteins that appear on the surface of healthy normal cells. However, unusually high-levels of high-mannose glycans are often found in cell-surface and secreted proteins produced by malignant cells.

Non-limiting examples of high-mannose-type glycans include: Man9GlcNAc2 (Man 9), Man8GlcNAc2 (Man8), Man7GlcNAc2 (Man 7), Man6GlcNAc2 (Man6) and Man5GlcNAc2 (Man5).

In some embodiments, the high-mannose-type glycan epitope is OVCA-associated (e.g., EOC-associated).

In particular embodiments, the high-mannose-type glycan epitope comprises one or more terminal α1,2-linked mannose residues.

In particular embodiments, the polypeptide specifically binds two or more high-mannose-type glycan epitopes, for example, 3, 4, 5 or more high-mannose-type glycan epitopes.

### Highly Glycosylated Proteins

In some embodiments, the polypeptide specifically binds a highly glycosylated protein.

In certain embodiments, the highly glycosylated protein comprises at least about 10 N-glycosylation sites, for example, at least about: 11, 12, 13, 14, 15, 16, 17 or 18 N-glycosylation sites. In particular embodiments, the highly glycosylated protein comprises about 13-16 N-glycosylation sites.

In some embodiments, the polypeptide specifically binds more than one highly glycosylated proteins, for example, 2, 3, 4, 5 or more highly glycosylated proteins.

### Cancer Cells

In some embodiments, the OVCA cell is an EOC cell.

In some embodiments, the OVCA cell (e.g., EOC cell) is an in vitro cell. In certain embodiments, the OVCA cell (e.g., EOC cell) is an ex vivo cell.

In some embodiments, the OVCA cell (e.g., EOC cell) is a cell of a subject described herein (e.g., a human patient). In certain embodiments, the OVCA cell (e.g., EOC cell) is a mammalian cell, e.g., a cell from a dog, a cat, a mouse, a rat, a hamster, a guinea pig, a horse, a pig, a sheep, a cow, a chimpanzee, a macaque, a cynomolgus, or a human. In some embodiments, the OVCA cell (e.g., EOC cell) is a mouse cell. In some embodiments, the OVCA cell (e.g., EOC cell) is a primate cell. In particular embodiments, the OVCA cell (e.g., EOC cell) is a human cell.

In some embodiments, the OVCA cell (e.g., EOC cell) is a cell of an adult human patient, for example, 18-75 years of age, 18 years of age or older, or 40 years of age or older. In certain embodiments, the OVCA cell (e.g., EOC cell) is a cell of a juvenile human patient. In some embodiments, the OVCA cell (e.g., EOC cell) is a cell of a human patient who is 18 years of age or younger, and/or 12 years of age or older. In particular embodiments, the OVCA cell (e.g., EOC cell) is a cell of a pediatric human patient.

In some embodiments, the OVCA cell (e.g., EOC cell) is a cell of a subject newly diagnosed with OVCA (e.g., EOC). In certain embodiments, the OVCA cell (e.g., EOC cell) is a cell of a subject who has been diagnosed with OVCA (e.g., EOC) for at least about 1 month, at least about 1 year, at least about 5 years, or at least about 10 years.

In certain embodiments, the OVCA cell (e.g., EOC cell) is a cell of an untreated subject (e.g., a human patient). In particular embodiments, the OVCA cell (e.g., EOC cell) is a cell of a subject who has received one or more prior anti-cancer therapies. In some embodiments, the OVCA cell (e.g., EOC cell) is a cell of a subject who has relapsed and/or refractory ovarian cancer.

In certain embodiments, the OVCA cell (e.g., EOC cell) is characterized by an abnormal surface accumulation of high-mannose glycans. In some embodiments, cell surface high-mannose glycans on an OVCA cell (e.g., an EOC cell) of the disclosure are at least about 2 times higher than on a normal cell, for example, at least about: 2.5, 3, 3.5, 4, 4.5, 5, 5.5, 6, 6.5, 7, 7.5, 8, 8.5, 9, 9.5 or 10 times higher than on a normal cell. In particular embodiments, about 2-10 times higher than on a normal cell, for example about: 2.5-10, 2.5-9.5, 2.5-9, 3-9, 3-8.5, 3.5-8.5, 3.5-8, 4-8, 4-7.5, 4.5-7, 4.5-6.5, 5-6.5, 5-6 or 5.5-6 times higher than on a normal cell.

In some embodiments, the OVCA cell (e.g., EOC cell) expresses a protein with an abnormal accumulation of high-mannose glycans on its cell surface. In particular embodiments, the OVCA cell (e.g., EOC cell) is characterized by one or more tumor-associated glycobiomarkers. In certain embodiments, the OVCA cell (e.g., EOC cell) is characterized by two or more tumor-associated glycobiomarkers.

### Methods of Treatment

In another aspect, the disclosure provides a method of treating OVCA in a subject in need thereof, the method comprises administering to the subject a therapeutically effective amount of a polypeptide comprising an actinohivin or a variant thereof.

In another aspect, the disclosure provides use of a polypeptide in manufacture of a medicament for treating OVCA in a subject in need thereof, wherein the polypeptide comprises an actinohivin or a variant thereof, and wherein the treatment comprises administering to the subject an effective amount of the polypeptide.

In another aspect, the disclosure provides a polypeptide for use in treating OVCA in a subject in need thereof, wherein the polypeptide comprises an actinohivin or a variant thereof, and wherein the treatment comprises administering to the subject an effective amount of the polypeptide.

In another aspect, the disclosure provides a method of reducing tumor size in a subject having OVCA, the method comprises administering to the subject an effective amount of a polypeptide comprising an actinohivin or a variant thereof, wherein the administering of the polypeptide reduces the tumor size.

In another aspect, the disclosure provides use of a polypeptide in manufacture of a medicament for reducing tumor size in a subject having OVCA, wherein the polypeptide comprises an actinohivin or a variant thereof, and wherein the treatment comprises administering to the subject an effective amount of the polypeptide.

In another aspect, the disclosure provides a polypeptide for use in reducing tumor size in a subject having OVCA, wherein the polypeptide comprises an actinohivin or a variant thereof, and wherein the treatment comprises administering to the subject an effective amount of the polypeptide.

The polypeptide comprising an actinohivin or a variant thereof may be any one of the polypeptides described herein.

In another aspect, the disclosure provides a method of treating OVCA (e.g., EOC) in a subject in need thereof, the method comprises administering to the subject an effective amount of a polypeptide comprising an amino acid sequence set forth in SEQ ID NO:16.

In another aspect, the disclosure provides use of a polypeptide in manufacture of a medicament for treating OVCA (e.g., EOC) in a subject in need thereof, wherein the polypeptide comprises an amino acid sequence set forth in SEQ ID NO:16, and wherein the method comprises administering to the subject an effective amount of the polypeptide.

In another aspect, the disclosure provides a polypeptide for use in treating OVCA (e.g., EOC) in a subject in need thereof, wherein the polypeptide comprises an amino acid sequence set forth in SEQ ID NO:16, and wherein the method comprises administering to the subject an effective amount of the polypeptide.

### Compositions

In some embodiments, a polypeptide described herein is provided in a composition, for example in a pharmaceutical composition.

In some embodiments, the composition (e.g., pharmaceutical composition) further comprises one or more pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the composition (e.g., pharmaceutical composition) of the disclosure is formulated for a suitable administration schedule and route. Non-limiting examples of administration routes include oral, rectal, mucosal, intravenous, intramuscular, subcutaneous and topical, etc. In some embodiments, the composition (e.g., pharmaceutical composition) of the disclosure is stored in the form of an aqueous solution or a dried formulation (e.g., lyophilized).

In some embodiments, the composition is formulated to be administered by infusion (e.g., intravenous infusion) or injection (e.g., intramuscular, subcutaneous, intraperitoneal or intratumoral injection). In certain embodiments, the composition is formulated to be administered by intravenous infusion. In some embodiments, the composition is formulated to be administered by intramuscular injection. In particular embodiments, the composition is formulated to be administered by subcutaneous injection. In some embodiments, the composition is formulated to be administered by intraperitoneal injection. In certain embodiments, the composition is formulated to be administered by intratumoral injection.

In some embodiments, the composition is formulated to be administered with one or more additional therapeutic agents as a combination therapy. Non-limiting examples of the one or more additional therapeutic agents include a T cell expressing chimeric antigen receptor (CAR) (CAR-T cell), a natural killer cell expressing CAR (CAR-NK cell), a macrophage expressing CAR (CAR-M cell), a chemotherapeutic agent, an immune checkpoint inhibitor, a T-cell redirector, radiation therapy, surgery and a standard of care drug.

In certain embodiments, the surgery comprises total abdominal hysterectomy, bilateral salpingo-oophorectomy, or omentectomy.

In some embodiments, the chemotherapeutic agent comprises cisplatin.

In certain embodiments, the chemotherapy comprises a platinum-based drug (e.g., carboplatin) and a taxane (e.g., paclitaxel).

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

Pharmaceutical composition (or pharmaceutical combination) referring to a product that results from combining a polypeptide that comprises an actinohivin or a variant thereof and one or more additional therapeutic agents includes both fixed and non-fixed combinations.

“Fixed combination” refers to a single pharmaceutical composition comprising two or more compounds, for example, the polypeptide that comprises an actinohivin or a variant thereof and the one or more additional therapeutic agents are administered simultaneously in the form of a single entity or dosage. In some embodiments, a pharmaceutical composition comprising the polypeptide that comprises an actinohivin or a variant thereof and the one or more additional therapeutic agents are provided as a fixed combination.

“Non-fixed combination” refers to separate pharmaceutical compositions, wherein each comprises one or more compounds, for example, the polypeptide that comprises an actinohivin or a variant thereof and the one or more additional therapeutic agents are administered as separate entities either simultaneously, concurrently or sequentially with no specific intervening time limits, wherein such administration provides effective levels of the two or more compounds in the body of the subject. In some embodiments, pharmaceutical composition comprising the polypeptide that comprises an actinohivin or a variant thereof and the one or more additional therapeutic agents are provided as a non-fixed combination.

In some embodiments, the polypeptide (e.g., AvFc) is systemically administered to the subject at about 10-50 mg/kg, for example, at about: 10 mg/kg, 15 mg/kg, 20 mg/kg, 25 mg/kg, 30 mg/kg, 35 mg/kg, 40 mg/kg, 45 mg/kg or 50 mg/kg, or at about: 10-45 mg/kg, 15-45 mg/kg, 15-40 mg/kg, 20-40 mg/kg, 20-35 mg/kg, 25-35 mg/kg or 25-30 mg/kg. In certain embodiments, the polypeptide (e.g., AvFc) is systemically administered to the subject at about 10-50 mg/kg about every 2-7 days (for example, about every: 2, 3, 4, 5, 6 or 7 days,) for about 2-10 weeks (for example, for about: 2, 3, 4, 5, 6, 7, 8, 9 or 10 weeks, or for about: 2-9, 3-9, 3-8, 4-8, 4-7, 5-7 or 5-6 weeks). In particular embodiments, the polypeptide (e.g., AvFc) is systemically administered to the subject at about 25 mg/kg of every other day (Q2D) for 14 or 20 days (8 or 11 doses total, respectively). In some embodiments, the polypeptide (e.g., AvFc) is systemically administered to the subject at about 10-50 mg/kg of every 7 days (Q7D) for 1-2 months.

In some embodiments, a polypeptide or pharmaceutical composition of the disclosure (e.g., AvFc) is administered in combination with a second therapeutic agent. In certain embodiments, the second therapeutic agent is an antibody (e.g., a monoclonal antibody (mAb)). The antibody can target an aspect of the cancer itself (such as a tumor-associated antigen) or a physiological/immunological process (such as checkpoint inhibitors, which prevent or delay T cell anergy and apoptosis), or targets endogenous VEGF and inhibits blood vessel formation in the tumor (e.g., bevacizumab). In particular embodiments, the second therapeutic agent comprises an adoptive immunotherapy agent, an antibody-drug conjugate, a chemotherapeutic agent, an immune checkpoint inhibitor, or a PARP inhibitor, or a combination thereof. A non-limiting example of a chemotherapy combination comprises a platinum-based drug (e.g., carboplatin) and a taxane (e.g., paclitaxel). Non-limiting examples of immune checkpoint inhibitors include pembrolizumab (KEYTUIDA®, Merck), durvalumab (IMFINZI®, Medimmune/AstraZeneca), dostarlimab (Tesaro), avelumab (Bavencio®, Merck KGaA/Pfizer), atezolizumab (TECENTRIQ®, Genentech/Roche), and nivolumab (OPDIVO®, Bristol-Myers Squibb). Non-limiting examples of PARP inhibitors include niraparib (ZEJULA®, Tesaro), olaparib (LYNPARZA®, AstraZeneca), and rucaparib (Rubraca®, Clovis Oncology), etc. A non-limiting example of antibody-drug conjugates is mirvetuximab soravtansine, which targets the human folate receptor 1 (FOLR1). A non-limiting example of an adoptive immunotherapy is Chimeric Antigen Receptor T cells (CAR-T cells), e.g., targeting FOLR1 or Cytokine-Induced Killer cells (CIK cells).

### Ovarian Cancer

“Cancer” refers to an abnormal growth of cells which tend to proliferate in an uncontrolled way and, in some cases, to metastasize (spread) to other areas of a patient's body.

In some embodiments, the OVCA is EOC.

In some embodiments, the OVCA (e.g., EOC) is characterized by one or more tumor-associated glycobiomarker. In certain embodiments, the OVCA (e.g., EOC) is characterized by two or more tumor-associated glycobiomarkers.

In certain embodiments, the OVCA (e.g., EOC) is characterized by an abnormal cell-surface accumulation of high-mannose glycans. In some embodiments, cell surface high-mannose glycans on an OVCA cell (e.g., an EOC cell) of the disclosure are about 2-10 times higher than on a normal cell.

In some embodiments, the OVCA (e.g., EOC) is characterized by cell-surface expression of a protein with an abnormal accumulation of high-mannose glycans.

In particular embodiments, the OVCA (e.g., EOC) is chemo-resistant.

### Subjects

The term “subject” refers to an animal (e.g., a mammal). In some embodiments, the subject is a mammal. In certain embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate. In particular embodiments, the subject is a human.

The terms “subject in need thereof” refers to a mammalian subject, preferably human, diagnosed with or suspected of having a disease (e.g., OVCA such as a EOC), whom will be or has been administered a polypeptide according to a method of the invention. “Subject in need thereof” includes those subjects already with the undesired physiological change or disease as well as those subjects prone to have the physiological change or disease.

Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the subject is an adult patient. In certain embodiments, the subject is a juvenile patient. In particular embodiments, the subject is a pediatric patient.

In some embodiments, the subject is 18-75 years of age. In certain embodiments, the subject is 40 years of age or older, e.g., at least: 45, 50, 55, 60, 65, 70, 75, 80, 85 or 90 years old.

In certain embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject is 12 years of age or older.

In certain embodiments, the subject is two years of age or older, for example, at least: 3, 4, 5, 6, 7, 8, 9, 10, 11, 12 years of age or older. In some embodiments, the subject is 4 years of age or older. In some embodiments, the subject is 5 years of age or older. In some embodiments, the subject is 6 years of age or older.

In some embodiments, the subject has been diagnosed with OVCA (e.g., EOC) for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years or 10 years.

In particular embodiments, the subject is newly diagnosed with OVCA (e.g., EOC). “Newly diagnosed” refers to a subject who has been diagnosed with OVCA (e.g., EOC) but has not yet received treatment for the OVCA.

In certain embodiments, the subject is treatment naïve.

In some embodiments, the subject has received one or more prior anti-cancer therapies. In particular embodiments, the one or more prior anti-cancer therapies comprises surgery, one or more chemotherapeutic agents, checkpoint inhibitors, targeted anti-cancer therapies or kinase inhibitors, or any combination thereof. In certain embodiments, the surgery comprises total abdominal hysterectomy, bilateral salpingo-oophorectomy, or omentectomy. In some embodiments, the chemotherapy comprises a platinum-based drug (e.g., carboplatin) and a taxane (e.g., paclitaxel).

A major issue in the current EOC management is the recurrence of chemo-resistant tumors. In particular embodiments, the subject has undergone primary therapy and achieved the no residual disease status.

In certain embodiments, a polypeptide of the disclosure (e.g., AvFc) is administered complement to, or as a replacement for, existing maintenance therapies in the absence of disease recurrence. For example, the polypeptide (e.g., AvFc) is formulated in a solution for intravenous (i.v.) infusion or intraperitoneal (i.p.) injection in an outpatient setting and is given to patients following completion of primary therapy. Administration of the drug in an outpatient clinic setting can also allow for close patient monitoring.

In some embodiments, a polypeptide of the disclosure (e.g., AvFc) is administered as a complement to, or a replacement for, existing second-line therapies in the event of disease recurrence. For example, the polypeptide (e.g., AvFc) is initially administered in an inpatient setting followed by administration in outpatient clinics for several weeks depending on patient condition, then followed by maintenance treatment in an outpatient setting as necessary.

In particular embodiments, the subject is relapsed or resistant to treatment with one or more prior anti-cancer therapies. “Refractory” refers to a disease that does not respond to a treatment. A refractory disease can be resistant to a treatment before or at the beginning of the treatment, or a refractory disease can become resistant during a treatment. “Relapsed” refers to the return of a disease or the signs and symptoms of a disease after a period of improvement after prior treatment with a therapeutic. In some embodiments, the subject has relapsed ovarian cancer. In certain embodiments, the subject has refractory ovarian cancer. In particular embodiments, the subject has relapsed and refractory ovarian cancer.

### Treating

“Treat”, “treating” or “treatment” of a disease or disorder such as OVCA refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, and weight of a mammal, mode of administration and the ability of a therapeutic, or combination of therapeutics, to elicit a desired response in an individual.

In some embodiments, the therapeutically effective amount of the polypeptide is sufficient to induce a cytotoxic effect. In certain embodiments, the cytotoxic effect comprises one or more Fc-mediated cytotoxic effects (e.g., ADCC).

In particular embodiments, the cytotoxic effect (e.g., ADCC) is induced (e.g., increased) by at least about 10%, for example, by at least about: 15%, 20%, 25%, 30%, 35%, 40%, 45%, 50%, 55%, 60%, 65%, 70%, 75%, 80%, 85% or 90%. In some embodiments, the cytotoxic effect (e.g., ADCC) is induced by about 1-90%, for example, by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In particular embodiments, the cytotoxic effect (e.g., ADCC) is induced by at least about 100%, for example, by at least about: 1.5-fold, 2-fold, 2.5-fold, 3-fold, 3.5-fold, 4-fold, 4.5-fold, 5-fold, 6-fold, 7-fold, 8-fold, 9-fold, 10-fold, 11-fold, 12-fold, 13-fold, 14-fold, or 15-fold. In some embodiments, the cytotoxic effect (e.g., ADCC) is induced by about 1-15 folds, for example, by about: 1-14 folds, 1.5-14 folds, 1.5-13 folds, 2-13 folds, 2-12 folds, 2.5-12 folds, 2.5-11 folds, 3-11 folds, 3-10 folds, 3.5-10 folds, 3.5-9 folds, 4-9 folds, 4-8 folds, 4.5-8 folds, 4.5-7 folds, 5-7 folds, or 5-6 folds.

In some embodiments, the therapeutically effective amount of the polypeptide is sufficient to inhibited (e.g., slowed and/or reduced) tumor growth. In certain embodiments, tumor growth is inhibited by at least about 10%, for example, by at least about: 15%, 20%, 25%, 30%, 35%, 40%, 45%, 50%, 55%, 60%, 65%, 70%, 75%, 80%, 85% or 90%. In some embodiments, tumor growth is inhibited by about 1-90%, for example, by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In particular embodiments, the therapeutically effective amount is sufficient to significantly decrease tumor burden, improve survival (e.g., extend survival and/or increase the likelihood of survival), or both.

“Survival” refers to the patient remaining alive and can be estimated by the Kaplan-Meier method. “Survival” includes progression free survival (PFS) and overall survival (OS).

“PFS” refers to the time from treatment to first disease progression or death. PFS can be assessed by, for example, Response Evaluation Criteria in Solid Tumors (RECIST). In some embodiments, the PFS is extended by at least about 1 month, for example, by at least about: 1.5, 2, 2.5, 3, 3.5, 4, 4.5, 5, 5.5, 6, 7, 8, 9, 10, 11, 12, 15, 18, 21 or 24 months compared to a control.

“OS” refers to the patient remaining alive for a defined period of time, such as about: 1, 1.5, 2, 3, 4, 5, or 10 years, from initiation of treatment or from initial diagnosis. In some embodiments, the OS is extended by at least about 1 month, for example, by at least about: 1.5, 2, 2.5, 3, 3.5, 4, 4.5, 5, 5.5, 6, 7, 8, 9, 10, 11, 12, 15, 18, 21 or 24 months compared to a control.

In some embodiments, the therapeutically effective amount of the polypeptide is sufficient to achieve no residual disease status (including any abdominal metastases), to prevent disease recurrence, or both.

### Diagnosis

In certain embodiments, methods of treatment further comprise determining if a biological sample of the subject in need is characterized with high-mannose-type glycan epitopes.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

In some embodiments, the method further comprises:
a) providing a biological sample from the subject; and
b) determining presence or absence of an abnormal accumulation of the high-mannose glycan epitope in the biological sample.

In some embodiments, the biological sample comprises an ovarian biopsy (e.g., an ovarian tumor biopsy). In certain embodiments, the biological sample comprises a blood sample.

While example embodiments have been particularly shown and described, it will be understood by those skilled in the art that various changes in form and details may be made therein without departing from the scope of the embodiments encompassed by the appended claims.

### Embodiments

A method of treating ovarian cancer in a subject in need thereof, comprising administering to the subject a therapeutically effective amount of a polypeptide comprising an actinohivin variant.

The method of Item 1, wherein the ovarian cancer is epithelial ovarian cancer.

The method of Item 1 or 2, wherein the subject has undergone primary therapy and achieved the no residual disease status.

The method of Item 1 or 2, wherein the subject has relapsed and refractory ovarian cancer.

The method of Item 1 or 2, wherein the ovarian cancer is chemo-resistant.

The method of any one of Items 1-5, wherein the therapeutically effective amount is sufficient to significantly decrease tumor burden, improve progression-free survival, improve overall survival, or a combination thereof.

The method of any one of Items 1-6, wherein the polypeptide is formulated for intravenous (IV) administration.

The method of any one of Items 1-7, wherein the polypeptide is administered in combination with a second therapeutic agent.

The method of any one of Items 1-8, wherein the subject is a human.

The method of Item 9, wherein the polypeptide is administered in a clinical outpatient setting.

The method of Item 9, wherein the polypeptide is administered in a clinical inpatient setting.

A method of killing an ovarian cancer cell, the method comprises contacting the ovarian cancer cell with a polypeptide comprising an actinohivin variant.

The method of Item 12, wherein the ovarian cancer cell is an epithelial ovarian cancer cell.

The method of Item 12 or 13, wherein the ovarian cancer cell is chemo-resistant.

The method of any one of Items 12-14, wherein the polypeptide induces killing of the ovarian cancer cell by antibody-dependent cell-mediated cytotoxicity (ADCC).

The method of any one of Items 12-15, further comprising contacting the ovarian cancer cell with a second therapeutic agent.

The method of any one of Items 1-16, wherein the actinohivin variant comprises an amino acid sequence selected from the group consisting of SEQ ID NOs:2-13, and optionally, the actinohivin variant comprises an amino acid sequence set forth in SEQ ID NO:9.

The method of any one of Items 1-17, wherein the polypeptide further comprises a fragment crystallizable domain of an antibody (Fc), and optionally, the polypeptide comprises an amino acid sequence set forth in SEQ ID NO:16.

The method of any one of Items 1-17, wherein the polypeptide further comprises a fragment antigen-binding domain of an antibody (Fab) or a single chain variable fragment of an antibody (scFv).

The method of any one of Items 1-19, wherein the polypeptide is modified or conjugated to a heterologous moiety, or a combination thereof.

### EXAMPLES

Ovarian cancer is the deadliest gynecological cancer. Ovarian cancer (OVCA), in particular epithelial ovarian cancer (EOC), is the deadliest gynecological cancer worldwide. Treatment of this disease includes a combination of surgery and chemotherapy with platinum-based drugs and taxanes. While most patients respond to the first-line therapies, nearly all will experience fatal recurrent disease—most often chemoresistant. Recent FDA approval of PARP inhibitors and bevacizumab as maintenance therapies has increased the progression-free survival period after primary therapy but has had little effect on overall survival rates.

The lack of novel or complementary second-line therapies, the lack of maintenance therapies that improve overall survival, and the increasing evidence that high-mannose glycans may be a unique and druggable target for EOC provide compelling arguments for the proposed product. Aberrant glycosylation of cancer-cell surfaces is a well-described phenomenon and is considered to be a hallmark of the disease. Recent advances in tumor glycobiology have demonstrated that various tumor types display an increased level of high-mannose glycans on their surface, and that these glycans may play a role in malignancy and metastasis. High-mannose glycans occur early in the N-glycosylation pathway in the endoplasmic reticulum and are typically processed by mannosidases and glycosyltransferases prior to leaving the secretory pathway, and thus are not typically found on the surface of the cell under normal conditions. However, quantitative N-glycan analysis by mass spectrometry with formalin-fixed, paraffin-embedded tissues show that high-mannose glycans are overexpressed on the surface of OVCA tumors. Additionally, high-mannose glycans were shown to be significantly elevated in the membrane glycoproteins of EOC cell lines compared to non-cancerous ovarian epithelial cells and may increase metastatic activity in SKOV3 cells. High-mannose glycans may be a useful EOC biomarker and a potentially druggable target.

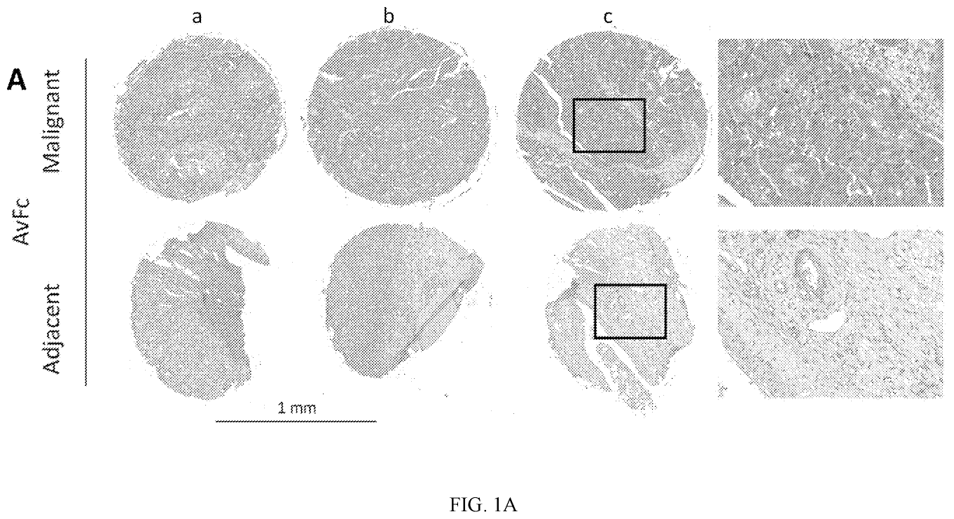

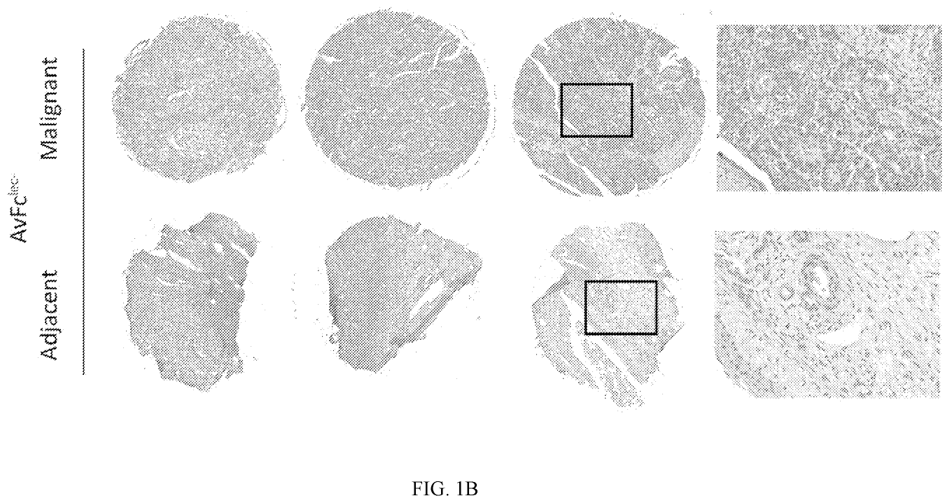

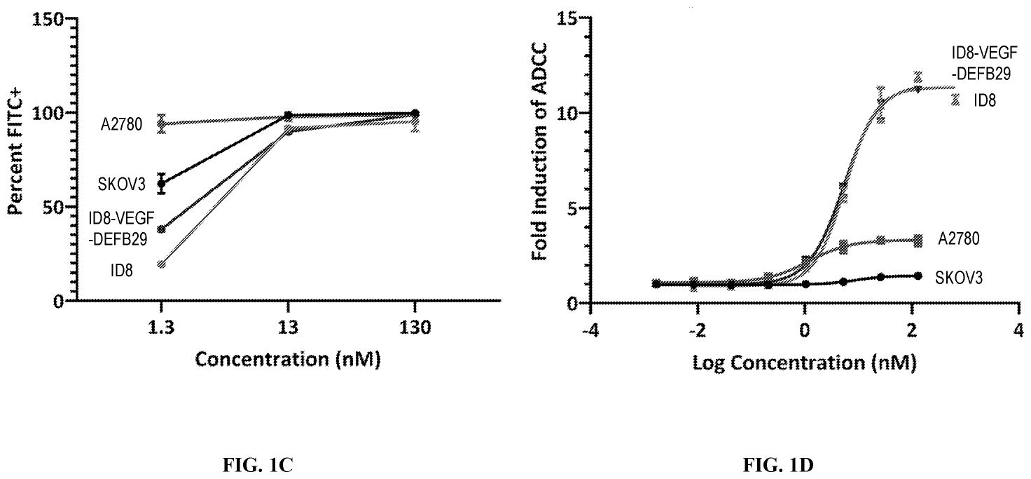

AvFc as a novel first-in-class targeted therapy for EOC. Avaren-Fc, is a potent antibody-like immunotherapeutic consisting of a high-mannose glycan-binding lectin fused to the Fc region of human IgG1 that is highly expressed and can be efficiently produced in Nicotiana benthamiana plants. Data described herein indicate that AvFc has a particularly high selectivity for EOC tissue and can bind to and potently induce antibody-dependent cell-mediated cytotoxicity (ADCC) against several EOC cell lines (FIGS. 1A-1C). Additionally, AvFc administration is well tolerated in multiple species including mice (both immunocompetent and deficient), rats, and rhesus macaques and is not cytotoxic or mitogenic to human peripheral blood mononuclear cells. It has been shown that repeated systemic administration of AvFc completely protected against hepatitis C virus challenge without causing any discernible toxicity in a human liver chimeric mouse model (hepatitis C virus also overexpresses high-mannose glycans on their surface). Based on these results concerning manufacturability, efficacy, and safety, AvFc may offer a powerful new option for EOC treatment by complementing or supplanting existing therapies for primary, secondary, or maintenance use. Such a therapy capable of improving overall survival in patients could potentially alter the paradigm of EOC management and introduce a new standard of care.

The vast majority of patients, regardless of stage, who receive first-line therapy (surgery and chemotherapy) for EOC will achieve no residual disease and subsequently begin maintenance therapy. Currently, maintenance therapy for EOC consists of the PARP inhibitors rucaparib, olaparib, or niraparib as well as the angiogenesis inhibitor bevacizumab. Clinical trials with these drugs have demonstrated significant improvements in progression-free survival but have yet to affect the overall survival rate. For instance, the ARIEL3 trial of rucaparib showed that the median progression-free survival in patients with BRCA-mutant EOC on rucaparib was 16.6 months, versus 5.4 months in the placebo control group. The results for bevacizumab compared to chemotherapy are similar (12.3 months versus 8.6 months median progression-free survival). Neither study reported complete overall survival data, but interim reporting in the OCEANS trial of bevacizumab did not demonstrate a significant improvement (median overall survival 35.2 months for chemotherapy compared to 33.3 months for chemotherapy plus bevacizumab). Upon failure of maintenance therapy and disease recurrence, the choice of secondary therapy depends on the platinum-free interval and an assessment of the surgical resectability of the tumor mass for secondary debulking surgery. For platin-sensitive disease, second-line therapies include platins, paclitaxel, liposomal doxorubicin, or gemcitabine. Unfortunately, recurrent EOC is most often chemo-resistant, particularly to the platins. In this setting, a number of sequential monochemotherapies, including paclitaxel, liposomal doxorubicin, or gemcitabine are used until subsequent disease progression or unacceptable toxicity occurs, which is all but inevitable. Thus, there is an urgent clinical need for novel or complementary therapies to cure the disease, prolong progression-free survival, and improve overall survival in patients.

Patients with late-stage EOC face poor prognosis and limited treatment options. Novel maintenance therapies like PARP inhibitors have increased progression-free survival but have yet to demonstrate benefit to overall survival. Additionally, second-line therapies are limited and generally ineffective. Furthermore, a recent analysis demonstrated that PARP inhibitors can be tremendously expensive and may not be cost-effective as maintenance therapies with incremental cost-effectiveness ratios of $235,000 and $287,000 per progression-free survival life-year. Therefore, any new intervention that improves progression-free survival and overall survival and would be welcomed.

As previously described, nearly all patients with EOC will develop recurrences despite achieving no residual disease after first-line therapy. Maintenance therapies can prolong time to recurrence but have thus far had little to no effect on survival rates, and secondary therapies are limited owing to the rampancy of chemo-resistant disease. Additionally, there is no immunotherapy currently approved for EOC that targets a specific tumor biomarker. Surgery, maintenance, and chemotherapy all exact a tremendous toll on patients and providers alike not just in terms of monetary costs but in physical and mental costs. Thus, a novel therapy that can complement or supplant current therapies and cure the disease, prolong progression-free survival, or improve overall survival in patients would be welcomed by the medical community.

The goal of this project is to develop a novel therapeutic for ovarian cancer (OVCA).

To this end, a novel antibody-like molecule called Avaren-Fc (AvFc), which consists of a high-mannose-glycan-binding lectin, Avaren, fused to the Fc region of human IgG1, has been developed for the treatment of epithelial ovarian cancer. High-mannose glycans are immature glycans that are found in unusually high proportions on the surface of ovarian cancer cells and thus may be a unique and druggable target. AvFc has shown high selectivity for ovarian cancer tissue and is capable of binding to and inducing antibody-dependent cell-mediated cytotoxicity (ADCC) against a number of ovarian cancer cell lines (both human and mouse). Additionally, Avaren-Fc is not cytotoxic or mitogenic and displays no overt toxicity in animals.

### Example 1. Activity of AvFc Against OVCA

Example 1 demonstrates that AvFc binds to human ovarian cancer tissue in a high-mannose glycan-dependent manner. Immunohistochemistry (IHC) was performed on a tissue array (US Biomax, Rockville, MD) (FIGS. 1A-1B), which contained 3 Stage I HGSOC tissues from a 48-year-old (column a), 72-year-old (column b), and a 55-year-old patient (column c) and three adjacent normal ovary tissues (below). Immunohistochemistry of human ovarian epithelial cancer tissue sections shows that while malignant tissues were highly bound by AvFc, little to no staining was seen in the adjacent tissues (FIG. 1A). Furthermore, when the malignant and adjacent tissues were stained with a non-sugar-binding mutant, AvFc^{lec-}, no binding was seen in either case (FIG. 1B). Accordingly, AvFc clearly delineates malignant from normal adjacent tissue, and AvFc's binding to human ovarian cancer tissue is high-mannose glycan-dependent.

Targeted immunotherapies have been tremendously successful for other cancer types. Immunohistochemical analysis of AvFc binding to human high-grade serous ovarian carcinoma tissue indicated that AvFc is highly selective for malignant tissue over non-malignant adjacent tissue (FIG. 1A). Conversely, an AvFc mutant deficient in high-mannose glycan-binding activity (AvFc^{lec-}) was incapable of binding to the tissue (FIG. 1B), illustrating that the binding of AvFc is high-mannose glycan-mediated and that tissue from human patients is indeed covered with clusters of high-mannose glycans that can be distinguished by AvFc. This selectivity combined with the lack of toxicity seen in multiple animal models indicates that despite the fact that AvFc does not target a specific pro-tumorigenic molecule (like EGFR), it is selective enough for tumor tissues to be efficacious.

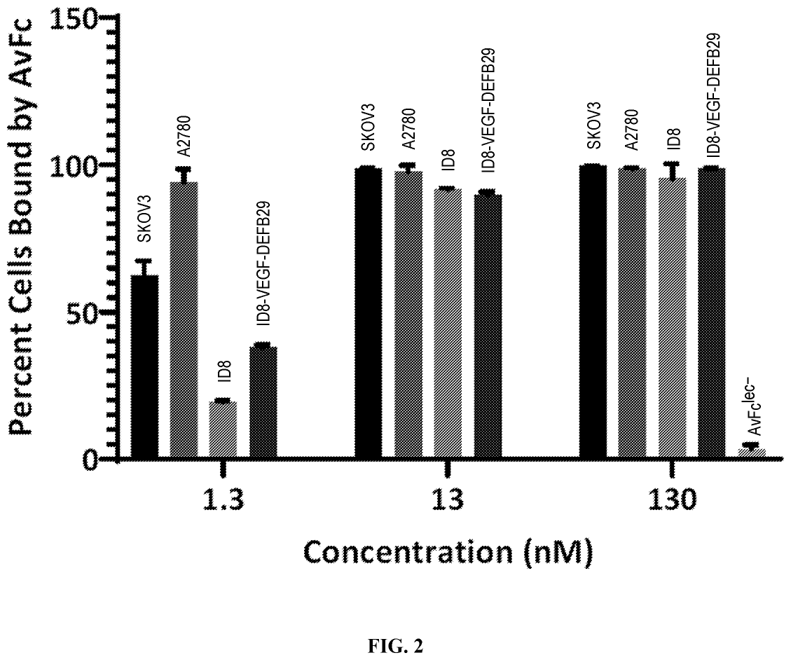

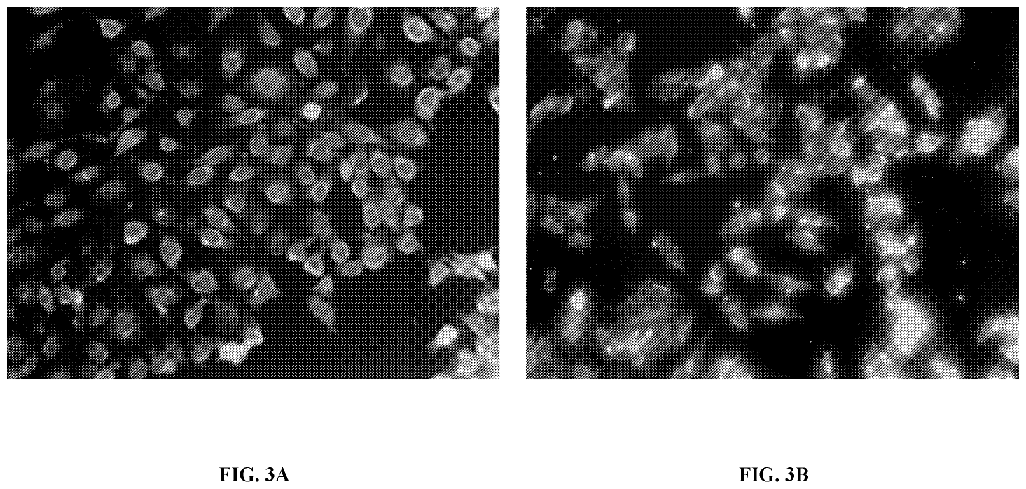

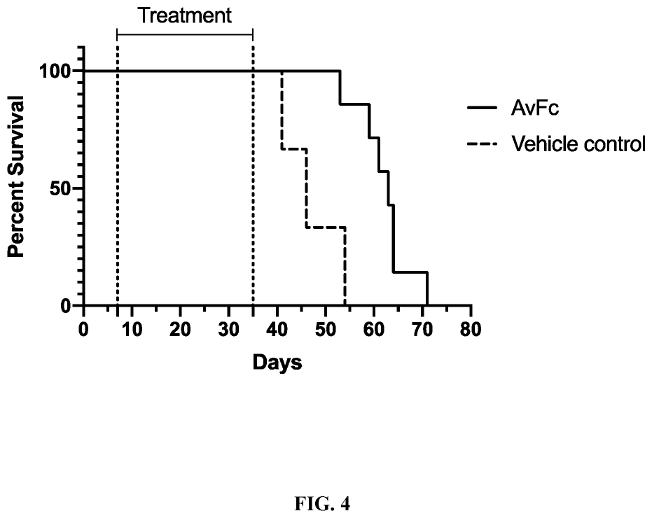

Binding of AvFc to a number of OVCA cell lines, including the human lines A2780 and SKOV3 and the murine lines ID8 and ID8-VEGF-DEFB29, was assessed by fluorescent staining (FIGS. 3A-3B) and flow cytometry (FIGS. 1C and 2). AvFc bound to these cell lines in a dose-dependent manner with saturation occurring at 13 nM (FIG. 2). The high level of binding suggests that AvFc would be a potent inducer of ADCC against these cancer cell lines. Indeed, in a luciferase-based reporter cell ADCC assay, AvFc was capable of inducing ADCC with EC₅₀ values in the low nanomolar range and with maximum fold induction values between 1.5-11.5-fold higher than the baseline (FIG. 1D and FIG. 4). AvFc may have significant in vivo activity against OVCA and justifies the use the ID8 mouse OVCA model.

### Example 2. Efficacy of AvFc in Mice

Female C57bl/6 mice were intraperitoneally (i.p.) challenged with two million ID8 cells per animal. Starting from 7 days post tumor challenge, mice were treated i.p. every 2 days with either 25 mg/kg AvFc or vehicle control for 28 days (Days 7-35; 15 doses total), and survival was monitored after treatment cessation until all animals died or reached a euthanasia point (>35 g body weight). Survival curves (FIG. 4) were determined to be significantly different by the long-rank test (P=0.0048; GraphPad Prism 8 software).

Additional in vivo data will be obtained to demonstrate the efficacy of AvFc in the ID8 murine EOC model. The efficacy of AvFc against EOC will be assessed using the murine ID8 EOC challenge model in immunocompetent mice. This model is a standard model in the field of ovarian cancer research and has been used for the assessment of therapeutic candidates. While testing AvFc's efficacy against human tumors may also be clinically relevant, currently available in vivo human tumor challenge models require the use of immunodeficient mice to successfully engraft and grow human-derived tumors in vivo. Here, since the primary proposed mechanism of action of AvFc requires a functional immune system, the proposed ID8 challenge study provides an appropriate model.

Study 1-1: ID8 challenge experiments. 4×10⁶ ID8-luc cells in PBS will be implanted intraperitoneally on day 0. Intraperitoneal treatment with AvFc will begin on day 7 post-implantation and continue Q2D for 28 days, with a dose level of 25 mg/kg or 10 mg/kg. This will be compared to the non-HMG-binding mutant version of AvFc, AvFc^{lec-}, as well as to cisplatin (5 mg/kg QW for 28 days) which has a history of efficacy in this model and can be used as a positive control. Disease progression will be monitored through weekly measurements of abdomen circumference and body weight as well as twice weekly measurements of bioluminescence by injecting 150 mg/kg luciferin and using in vivo live animal imaging. Animals will be euthanized when reaching 35 g or when moribund. Following euthanasia, immune cells will be collected by peritoneal lavage, and immunophenotyping will be conducted by flow cytometry to assess if AvFc affects the composition of the immune cells in the tumor microenvironment.

Results of the ID8 challenge model will be assessed. AvFc is expected to significantly increase survival and significantly decrease tumor burden as determined by abdominal circumference and bioluminescence. The model will be used to assess combination of AvFc treatment with cisplatin (which represents a standard-of-care chemotherapy for EOC and has been shown to provide efficacy in the proposed model) to test for any potential additive or synergistic effects.

### Example 3. Optimization of AvFc

Rationale: Changes to the Fc region of an antibody can modify its affinity to the various Fc receptors (FcγRs) and affect its PK/PD properties. The use of antibody-drug conjugates (ADCs) may further improve the therapeutic activity of AvFc.

Study 2-1: Assessing impacts of AvFc Fc modifications on activity in vitro. The impacts of Fc modifications, in particular the GASDALIE and GnGn modifications, will be assessed on AvFc's activity using an in vitro reporter-based ADCC assay targeting a number of human ovarian cancer cell lines. Modified forms of AvFc will be compared to wild type plant-produced AvFc as well as AvFc produced in CHO cells.

Study 2-2: Assessing impacts of AvFc Fc modifications on activity in vivo. After assessing the impact of Fc modifications in vitro, the changes will be confirmed using the ID8 challenge model as described in Study 1-1.

Study 2-3: Explore the use of AvFc as an ADC carrier. ADCs based on AvFc and various OVCA chemotherapeutics (such as paclitaxel) will be produced and their effects will be examined using in vitro cytotoxicity assays as well as primary cell ADCC assays.

Milestone 3: The most effective form of AvFc will be used to carry on to potential GLP-toxicology studies as well as in-human studies.

Current chemotherapeutics have yet to drastically improve progression-free survival and overall survival in EOC patients, despite the tremendous cost. The use of a novel, first-in-class drug targeting a unique biomarker like AvFc has the potential to greatly improve patient outcomes. Complementing, synergizing with existing treatments, or replacing them outright would have a large impact of EOC patient care.

The target population for AvFc include patients diagnosed with any stage of EOC and who have completed the first-line standard of care, achieving no residual disease. Currently, it is estimated that 67,000 people are living with OVCA in the United States, and globally this number may be as high as 762,000 people. In 2018 it was estimated that there were 22,240 new cases of OVCA diagnosed in the United States, with an average overall incidence of 11.8 per 100,000. EOC is by far the most common form of OVCA, making up around 90% of all new OVCA diagnoses. The vast majority of these patients will achieve no residual disease after first-line treatments, however nearly all of them will experience disease recurrence within a year. While the incidence and mortality rates of OVCA have decreased in the last few decades and are still trending downward, the trend is weak and OVCA is projected to be a major cause of gynecological cancer-related mortality for women for the foreseeable future.

AvFc is a first-in-class immunotherapeutic antibody-like molecule that is highly selective for OVCA-derived high-mannose glycans and whose primary mechanism of action include antibody-dependent cell-mediated cytotoxicity (ADCC). As a novel first-in-class agent making use of a unique biomarker—high-mannose glycans—AvFc represents a new paradigm in OVCA immunotherapy. To date, there are no other high-mannose glycan-binding agents in clinical or pre-clinical development for this indication, as such AvFc stands out among the field of chemotherapeutics and biologics being developed. Furthermore, the lack of specific tumor-associated antigens identified for OVCA means that AvFc is one of few immunotherapies in development that can directly bind and kill OVCA cells.

### Sequences

SEQ ID NO:1 is an amino acid sequence of a wild-type actinohivin polypeptide

(SEQ ID NO: 1)
ASVTIRNAQTGRLLDSNYNGNVYTLPANGGNYQRWTGPGDGTVRNAQTG
RCLDSNYDGAVYTLPCNGGSYQKWLFYSNGYIQNVETGRVLDSNYNGNV
YTLPANGGNYQKWYTG

SEQ ID NO:2 is an amino acid sequence of an actinohivin variant polypeptide made in accordance with the presently-disclosed subject matter and designated herein as variant 1.

(SEQ ID NO: 2)
ASGTIRNAETGRLLDSNYDGAVYTLPANGGSYQRWTGPGDGTVRNAETG
RLLDSNYDGAVYTLPANGGSYQKWTGPGDGTIQNAETGRLLDSNYDGAV
YTLPANGGSYQKWTG

SEQ ID NO:3 is an amino acid sequence of another actinohivin variant polypeptide made in accordance with the presently-disclosed subject matter and designated herein as variant

(SEQ ID NO: 3)
ASGTIRNAETGRCLDSNYDGAVYTLPCNGGSYQRWTGPGDGTVRNAETG
RCLDSNYDGAVYTLPCNGGSYQKWTGPGDGTIQNAETGRCLDSNYDGAV
YTLPCNGGSYQKWTG

SEQ ID NO:4 is an amino acid sequence of another actinohivin variant polypeptide made in accordance with the presently-disclosed subject matter and designated herein as variant 3.

(SEQ ID NO: 4)
ASVTIRNAETGRLLDSNYNGNVYTLPANGGNYQRWTGPGDGTVRNAETG
RCLDSNYDGAVYTLPCNGGSYQKWLFYSNGYIQNVETGRVLDSNYNGNV
YTLPANGGNYQKWYTG

SEQ ID NO:5 is an amino acid sequence of another actinohivin variant polypeptide made in accordance with the presently-disclosed subject matter and designated herein as variant 4.

(SEQ ID NO: 5)
ASVTIRNAETGRCLDSNYNGNVYTLPCNGGNYQRWTGPGDGTVRNAETG
RCLDSNYDGAVYTLPCNGGSYQKWLFYSNGYIQNVETGRCLDSNYNGNV
YTLPCNGGNYQKWYTG

SEQ ID NO:6 is an amino acid sequence of another actinohivin variant polypeptide made in accordance with the presently-disclosed subject matter and designated herein as variant 5.

(SEQ ID NO: 6)
ASGTIRNAETGRLLDSNYNGNVYTLPANGGNYQRWTGPGDGTVRNAETG
RCLDSNYDGAVYTLPCNGGSYQKWTGPGDGTIQNAETGRVLDSNYNGNV
YTLPANGGNYQKWTG

SEQ ID NO:7 is an amino acid sequence of another actinohivin variant polypeptide made in accordance with the presently-disclosed subject matter and designated herein as variant 6.

(SEQ ID NO: 7)
ASGTIRNAETGRCLDSNYDGNVYTLPCNGGSYQRWTGPGDGTVRNAETG
RCLDSNYDGNVYTLPCNGGSYQKWTGPGDGTIQNAETGRCLDSNYDGNV
YTLPCNGGSYQKWTG

SEQ ID NO:8 is an amino acid sequence of another actinohivin variant polypeptide made in accordance with the presently-disclosed subject matter and designated herein as variant 7.

(SEQ ID NO: 8)
ASGTIRNAQTGRCLDSNYNGNVYTLPCNGGNYQRWTGPGDGTVRNAQTG
RCLDSNYDGAVYTLPCNGGSYQKWTGPGDGTIQNAETGRCLDSNYNGNV
YTLPCNGGNYQKWTG

SEQ ID NO:9 is an amino acid sequence of another actinohivin variant polypeptide made in accordance with the presently-disclosed subject matter and designated herein as variant 8 or Avaren (actinohivin variant expressed in Nicotiana).

(SEQ ID NO: 9)
ASGTIRNAETGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTG

SEQ ID NO:10 is an amino acid sequence of another actinohivin variant polypeptide made in accordance with the presently-disclosed subject matter and designated herein as variant 9.

(SEQ ID NO: 10)
ASGTIRNAQTGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTG

SEQ ID NO:11 is an amino acid sequence of another actinohivin variant polypeptide made in accordance with the presently-disclosed subject matter and designated herein as variant 10.

(SEQ ID NO: 11)
ASGTIRNAETGRCLDSNYNGNVYTLPCNGGNYQRWTGPGDGTVRNAQTG
RCLDSNYDGAVYTLPCNGGSYQKWTGPGDGTIQNAETGRCLDSNYNGNV
YTLPCNGGNYQKWTG

SEQ ID NO:12 is an amino acid sequence of another actinohivin variant polypeptide made in accordance with the presently-disclosed subject matter and designated herein as variant 11.

(SEQ ID NO: 12)
ASGTIRNAQTGRLLDSNYNGNVYTLPANGGNYQRWTGPGDGTVRNAQTG
RLLDSNYNGNVYTLPANGGNYQKWTGPGDGTIQNAQTGRVLDSNYNGNV
YTLPANGGNYQKWTG

SEQ ID NO:13 is an amino acid sequence of another actinohivin variant polypeptide made in accordance with the presently-disclosed subject matter and designated herein as variant 12.

(SEQ ID NO: 13)
ASGTIRNAETGRLLDSNYNGNVYTLPANGGNYQRWTGPGDGTVRNAETG
RLLDSNYNGNVYTLPANGGNYQKWTGPGDGTIQNAETGRVLDSNYNGNV
YTLPANGGNYQKWTG

SEQ ID NO:14 is an amino acid sequence of another actinohivin variant polypeptide made in accordance with the presently-disclosed subject matter and designated herein as variant 13.

(SEQ ID NO: 14)
ASGTIRNAETGRCLDSNYNGNVYTLPCNGGNYQRWTGPGDGTVRNAETG
RCLDSNYNGNVYTLPCNGGNYQKWTGPGDGTIQNAETGRCLDSNYNGNV
YTLPCNGGNYQKWTG

SEQ ID NO:15 is an amino acid sequence of another actinohivin variant polypeptide made in accordance with the presently-disclosed subject matter and designated herein as variant 14.

(SEQ ID NO: 15)
ASGTIRNAETGRCLDSNYDGNVYTLPCNGGNYQRWTGPGDGTVRNAETG
RCLDSNYDGNVYTLPCNGGNYQKWTGPGDGTIQNAETGRCLDSNYDGNV
YTLPCNGGNYQKWTG

SEQ ID NO:16 is an amino acid sequence including the actinohivin variant polypeptide of SEQ ID NO:9 (Variant 8) fused, via a linker polypeptide, to an amino acid sequence comprising the fragment crystallizable (Fc) region of immunoglobulin (Ig) G, and referred to herein as AvFc.

(SEQ ID NO: 16)
ASGTIRNAETGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTGGGGSVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

### REFERENCES

1. Lengyel, E., Ovarian cancer development and metastasis. The American journal of pathology, 2010. 177(3): p. 1053-1064.
2. Surveillance Research Program National Cancer Institute. SEER*Explorer: An interactive website for SEER cancer statistics [Internet]. [cited 2020 Apr. 2]; Available from: seer.cancer.gov/explorer/.
3. Lheureux, S., et al., Epithelial ovarian cancer. The Lancet, 2019. 393(10177): p. 1240-1253.
4. Moore, K., et al., Maintenance olaparib in patients with newly diagnosed advanced ovarian cancer. New England Journal of Medicine, 2018. 379(26): p. 2495-2505.
5. Coleman, R. L., et al., Bevacizumab and paclitaxel-carboplatin chemotherapy and secondary cytoreduction in recurrent, platinum-sensitive ovarian cancer (NRG Oncology/Gynecologic Oncology Group study GOG-0213): a multicentre, open-label, randomised, phase 3 trial. The Lancet Oncology, 2017. 18(6): p. 779-791.
6. Mirza, M. R., et al., Niraparib maintenance therapy in platinum-sensitive, recurrent ovarian cancer. New England Journal of Medicine, 2016. 375(22): p. 2154-2164.
7. Aghajanian, C., et al., OCEANS: a randomized, double-blind, placebo-controlled phase III trial of chemotherapy with or without bevacizumab in patients with platinum-sensitive recurrent epithelial ovarian, primary peritoneal, or fallopian tube cancer. Journal of clinical oncology, 2012. 30(17): p. 2039.
8. Coleman, R. L., et al., Rucaparib maintenance treatment for recurrent ovarian carcinoma after response to platinum therapy (ARIEL3): a randomised, double-blind, placebo-controlled, phase 3 trial. The Lancet, 2017. 390(10106): p. 1949-1961.
9. Hamanishi, J., et al., Safety and antitumor activity of anti-PD-1 antibody, nivolumab, in patients with platinum-resistant ovarian cancer. Journal of Clinical Oncology, 2015. 33(34): p. 4015-4022.
10. Colombo, I., et al., Immunologic and genomic characterization of high grade serous ovarian cancer (HGSOC) in patients (pts) treated with pembrolizumab (Pembro) in the phase II INSPIRE trial. 2017, American Society of Clinical Oncology.
11. Pearce, O. M., Cancer glycan epitopes: biosynthesis, structure and function. Glycobiology, 2018. 28(9): p. 670-696.
12. Oliveira-Ferrer, L., K. Legler, and K. Milde-Langosch. Role of protein glycosylation in cancer metastasis. in Seminars in cancer biology. 2017. Elsevier.
13. Loke, I., et al., Emerging roles of protein mannosylation in inflammation and infection. Molecular aspects of medicine, 2016. 51: p. 31-55.
14. Everest-Dass, A. V., et al., N-glycan MALDI imaging mass spectrometry on formalin-fixed paraffin-embedded tissue enables the delineation of ovarian cancer tissues. Molecular & Cellular Proteomics, 2016. 15(9): p. 3003-3016.
15. Zhang, X., et al., Discovery of specific metastasis-related N-glycan alterations in epithelial ovarian cancer based on quantitative glycomics. PLoS One, 2014. 9(2): p. e87978.
16. Anugraham, M., et al., Specific glycosylation of membrane proteins in epithelial ovarian cancer cell lines: glycan structures reflect gene expression and DNA methylation status. Molecular & Cellular Proteomics, 2014. 13(9): p. 2213-2232.
17. Hamorsky, K. T., et al., Engineering of a Lectibody Targeting High-Mannose-Type Glycans of the HIV Envelope. Molecular Therapy, 2019. 27(11): p. 2038-2052.
18. Dent, M., et al., Safety and Efficacy of Avaren-Fc Lectibody Targeting HCV High-Mannose Glycans in a Human Liver Chimeric Mouse Model. Cellular and molecular gastroenterology and hepatology, 2021. 11(1): p. 185-198.
19. Zhong, L., et al., Cost-Effectiveness of Niraparib and Olaparib as Maintenance Therapy for Patients with Platinum-Sensitive Recurrent Ovarian Cancer. J Manag Care Spec Pharm, 2018. 24(12): p. 1219-1228.
20. Bray, F., et al., Global cancer statistics 2018: GLOBOCAN estimates of incidence and mortality worldwide for 36 cancers in 185 countries. CA: A Cancer Journal for Clinicians, 2018. 68(6): p. 394-424.
21. Torre, L. A., et al., Ovarian cancer statistics, 2018. CA: A Cancer Journal for Clinicians, 2018. 68(4): p. 284-296.
22. Ghisoni, E., et al., Ovarian Cancer Immunotherapy: Turning up the Heat. International Journal of Molecular Sciences, 2019. 20(12): p. 2927.
23. Richardson, D. L., S. M. Seward, and K. N. Moore, Antibody Drug Conjugates in the Treatment of Epithelial Ovarian Cancer. Hematology/Oncology Clinics of North America, 2018. 32(6): p. 1057-1071.
24. ImmunoGen Inc. ImmunoGen Announces Top-Line Results from Phase 3 FORWARD I Study of Mirvetuximab Soravtansine in Ovarian Cancer. 2019 [cited 2020 Apr. 13]; Available from: http://investor.immunogen.com/news-releases/news-release-details/immunogen-announces-top-line-results-phase-3-forward-i-study.
25. Sertkaya, A., et al., Key cost drivers of pharmaceutical clinical trials in the United States. Clin Trials, 2016. 13(2): p. 117-26.
26. Wei, H., et al., Combinatorial PD-1 blockade and CD137 activation has therapeutic efficacy in murine cancer models and synergizes with cisplatin. PloS one, 2013. 8(12): p. e84927-e84927.
27. Guo, Z., et al., PD-1 blockade and OX40 triggering synergistically protects against tumor growth in a murine model of ovarian cancer. PloS one, 2014. 9(2).
28. van Brummelen, E. M., et al., Antidrug antibody formation in oncology: clinical relevance and challenges. The oncologist, 2016. 21(10): p. 1260.
29. Choi, Y., et al., Studying cancer immunotherapy using patient-derived xenografts (PDXs) in humanized mice. Experimental & Molecular Medicine, 2018. 50(8): p. 99.
30. BCC Publishing. Therapies and Diagnostics for Ovarian Cancer: Global Markets to 2022. 2018; Available from: www.bccresearch.com/market-research/healthcare/therapies-and-diagnostics-for-ovarian-cancer-report.html.
31. Chiba H. et al., Actinohivin, a novel anti-HIV protein from an actinomycete that inhibits syncytium formation: isolation, characterization, and biological activities. Biochem Biophys Res Commun. 2001 Mar. 30; 282(2):595-601.
32. Ahmed A A et al., Structural characterization of GASDALIE Fc bound to the activating Fc receptor FcγRIIIa. J Struct Biol. 2016 April; 194(1):78-89.
33. Strasser R et al., Generation of glyco-engineered Nicotiana benthamiana for the production of monoclonal antibodies with a homogeneous human-like N-glycan structure. Plant Biotechnol J. 2008 May; 6(4):392-402.
34. Gaudinski et al., VRC 606 Study Team. Safety and pharmacokinetics of the Fc-modified HIV-1 human monoclonal antibody VRC01LS: A Phase 1 open-label clinical trial in healthy adults. PLoS Med. 2018 Jan. 24; 15(1):e1002493.

The teachings of all patents, published applications and references cited herein are incorporated by reference in their entirety.

1. A method of treating ovarian cancer in a subject in need thereof, comprising administering to the subject a therapeutically effective amount of a polypeptide comprising an actinohivin or a variant thereof.

2. The method of claim 1, wherein the ovarian cancer is epithelial ovarian cancer.

3. The method of claim 1, wherein the subject has undergone primary therapy and achieved the no residual disease status.

4. The method of claim 1, wherein the subject has relapsed or refractory ovarian cancer.

5. The method of claim 1, wherein the ovarian cancer is chemo-resistant.

6. The method of claim 1, wherein the therapeutically effective amount is sufficient to decrease tumor burden, improve progression-free survival, improve overall survival, or a combination thereof.

7. The method of claim 1, wherein the polypeptide is formulated for intravenous (i.v.) administration.

8. The method of claim 1, wherein the polypeptide is administered in combination with a second therapeutic agent.

9. The method of claim 1, wherein the subject is a human.

10. The method of claim 9, wherein the polypeptide is administered in a clinical outpatient setting.

11. The method of claim 9, wherein the polypeptide is administered in a clinical inpatient setting.

12. A method of killing an ovarian cancer cell, the method comprises contacting the ovarian cancer cell with a polypeptide comprising an actinohivin or a variant thereof.

13. The method of claim 12, wherein the ovarian cancer cell is an epithelial ovarian cancer cell.

14. The method of claim 12, wherein the ovarian cancer cell is chemo-resistant.

15. The method of claim 12, wherein the polypeptide induces killing of the ovarian cancer cell by antibody-dependent cell-mediated cytotoxicity (ADCC).

16. The method of claim 12, further comprising contacting the ovarian cancer cell with a second therapeutic agent.

17. The method of claim 1, wherein the polypeptide comprises an amino acid sequence selected from the group consisting of SEQ ID NOs:1-15, and optionally, the actinohivin variant comprises an amino acid sequence set forth in SEQ ID NO:9.

18. The method of claim 1, wherein the polypeptide further comprises a fragment crystallizable domain of an antibody (Fc), and optionally, the polypeptide comprises an amino acid sequence set forth in SEQ ID NO:16.

19. The method of claim 1, wherein the polypeptide further comprises a fragment antigen-binding domain of an antibody (Fab) or a single chain variable fragment of an antibody (scFv).

20. The method of claim 1, wherein the polypeptide is modified or conjugated to a heterologous moiety, or a combination thereof.


SEQUENCE LISTING (16 sequences)

SEQ ID NO: 1 - protein, 114 aa, Artificial Sequence
  Actinomycete strain K97-0003
ASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 2 - protein, 113 aa, Artificial Sequence
  actinohivin variant polypeptide - Variant 1
ASGTIRNAETGRLLDSNYDGAVYTLPANGGSYQRWTGPGDGTVRNAETGRLLDSNYDGAVYTLPANGGSYQKWTGPGDGTIQNAETGRLLDSNYDGAVYTLPANGGSYQKWTG

SEQ ID NO: 3 - protein, 113 aa, Artificial Sequence
  actinohivin variant polypeptide - Variant 2
ASGTIRNAETGRCLDSNYDGAVYTLPCNGGSYQRWTGPGDGTVRNAETGRCLDSNYDGAVYTLPCNGGSYQKWTGPGDGTIQNAETGRCLDSNYDGAVYTLPCNGGSYQKWTG

SEQ ID NO: 4 - protein, 114 aa, Artificial Sequence
  actinohivin variant polypeptide - Variant 3
ASVTIRNAETGRLLDSNYNGNVYTLPANGGNYQRWTGPGDGTVRNAETGRCLDSNYDGAVYTLPCNGGSYQKWLFYSNGYIQNVETGRVLDSNYNGNVYTLPANGGNYQKWYTG

SEQ ID NO: 5 - protein, 114 aa, Artificial Sequence
  actinohivin variant polypeptide - Variant 4
ASVTIRNAETGRCLDSNYNGNVYTLPCNGGNYQRWTGPGDGTVRNAETGRCLDSNYDGAVYTLPCNGGSYQKWLFYSNGYIQNVETGRCLDSNYNGNVYTLPCNGGNYQKWYTG

SEQ ID NO: 6 - protein, 113 aa, Artificial Sequence
  actinohivin variant polypeptide - Variant 5
ASGTIRNAETGRLLDSNYNGNVYTLPANGGNYQRWTGPGDGTVRNAETGRCLDSNYDGAVYTLPCNGGSYQKWTGPGDGTIQNAETGRVLDSNYNGNVYTLPANGGNYQKWTG

SEQ ID NO: 7 - protein, 113 aa, Artificial Sequence
  actinohivin variant polypeptide - Variant 6
ASGTIRNAETGRCLDSNYDGNVYTLPCNGGSYQRWTGPGDGTVRNAETGRCLDSNYDGNVYTLPCNGGSYQKWTGPGDGTIQNAETGRCLDSNYDGNVYTLPCNGGSYQKWTG

SEQ ID NO: 8 - protein, 113 aa, Artificial Sequence
  actinohivin variant polypeptide - Variant 7
ASGTIRNAQTGRCLDSNYNGNVYTLPCNGGNYQRWTGPGDGTVRNAQTGRCLDSNYDGAVYTLPCNGGSYQKWTGPGDGTIQNAETGRCLDSNYNGNVYTLPCNGGNYQKWTG

SEQ ID NO: 9 - protein, 113 aa, Artificial Sequence
  Variant 8  Avaren (actinohivin variant expressed in Nicotiana)
ASGTIRNAETGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTG

SEQ ID NO: 10 - protein, 113 aa, Artificial Sequence
  actinohivin variant polypeptide - Variant 9
ASGTIRNAQTGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTG

SEQ ID NO: 11 - protein, 113 aa, Artificial Sequence
  actinohivin variant polypeptide - Variant 10
ASGTIRNAETGRCLDSNYNGNVYTLPCNGGNYQRWTGPGDGTVRNAQTGRCLDSNYDGAVYTLPCNGGSYQKWTGPGDGTIQNAETGRCLDSNYNGNVYTLPCNGGNYQKWTG

SEQ ID NO: 12 - protein, 113 aa, Artificial Sequence
  actinohivin variant polypeptide - Variant 11
ASGTIRNAQTGRLLDSNYNGNVYTLPANGGNYQRWTGPGDGTVRNAQTGRLLDSNYNGNVYTLPANGGNYQKWTGPGDGTIQNAQTGRVLDSNYNGNVYTLPANGGNYQKWTG

SEQ ID NO: 13 - protein, 113 aa, Artificial Sequence
  actinohivin variant polypeptide - Variant 12
ASGTIRNAETGRLLDSNYNGNVYTLPANGGNYQRWTGPGDGTVRNAETGRLLDSNYNGNVYTLPANGGNYQKWTGPGDGTIQNAETGRVLDSNYNGNVYTLPANGGNYQKWTG

SEQ ID NO: 14 - protein, 113 aa, Artificial Sequence
  actinohivin variant polypeptide - Variant 13
ASGTIRNAETGRCLDSNYNGNVYTLPCNGGNYQRWTGPGDGTVRNAETGRCLDSNYNGNVYTLPCNGGNYQKWTGPGDGTIQNAETGRCLDSNYNGNVYTLPCNGGNYQKWTG

SEQ ID NO: 15 - protein, 113 aa, Artificial Sequence
  actinohivin variant polypeptide - Variant 14
ASGTIRNAETGRCLDSNYDGNVYTLPCNGGNYQRWTGPGDGTVRNAETGRCLDSNYDGNVYTLPCNGGNYQKWTGPGDGTIQNAETGRCLDSNYDGNVYTLPCNGGNYQKWTG

SEQ ID NO: 16 - protein, 350 aa, Artificial Sequence
  actinohivin variant 8 fused to human IgG1 Fc (AvFc)
ASGTIRNAETGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTGGGGSVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK